(19) United States
(12) Patent Application Publication
(10) Pub. No.: US 2023/0130186 A1
(43) Pub. Date: Apr. 27, 2023

(54) RAPID POSITIONING SYSTEMS

(71) Applicant: CENSYN, INC., LAKE FOREST, CA (US)

(72) Inventors: Ayushi Hitesh PATEL, Irvine, CA (US); Trevor Gray SILENCE, Duarte, CA (US); Anirudh Poomima BHUSHAN, Lake Forest, CA (US)

(21) Appl. No.: 18/087,568

(22) Filed: Dec. 22, 2022

Related U.S. Application Data
(63) Continuation-in-part of application No. PCT/US2021/039533, filed on Jun. 29, 2021.
(60) Provisional application No. 63/047,192, filed on Jul. 1, 2020.
(60) Provisional application No. 63/084,390, filed on Sep. 28, 2020.

(51) Int. Cl. A61B 5/00; A61B 5/25

(52) U.S. Cl. CPC A61B 5/6841; A61B 5/25; A61B 5/7425; A61B 2562/0219; A61B 2560/0238

(57) ABSTRACT

Provided are systems and methods for tracking one or more electrode positions.

### CROSS-REFERENCE

This application claims the benefit of PCT Application No. PCT/US21/039533 filed Jun. 29, 2021 which claims the benefit of U.S. Provisional Application No. 63/047,192, filed Jul. 1, 2020, and further claims benefit of U.S. Provisional Application No. 63/084,390, filed Sep. 28, 2020, in which all of these applications are incorporated herein by reference.

### SUMMARY

Provided herein are embodiments of a system to guide placement of one or more electrodes on a surface of a subject to detect bioelectric signals, the system comprising: one or more inertial sensors coupled with the one or more electrodes; and a computing device comprising a processor in operative communication with the one or more inertial sensors, and a non-transitory computer readable storage medium having a computer program with instructions executable by the processor, such that the processor is configured to: calibrate a first position of the one or more electrodes to set an origin location, receive data from the one or more inertial sensors to track a second position of the one or more electrodes relative to the origin location, and output a feedback corresponding to the second position of the one or more electrodes.

In some embodiments, the system further comprises a camera in operative communication with the processor, and the processor is configured to receive data from the camera, via the computer program instructions, such that the processor is configured to track the second position of the one or more electrodes and output the feedback based on the one or more inertial sensors and the camera. In some embodiments, the processor, via the computer program instructions, is configured to detect at least one edge of the surface from one or more images captured by the camera, and track the second position of the one or more electrodes based on a position of the at least one edge in the one or more images.

In some embodiments, the system further comprises one or more force sensors to detect a force being applied by the one or more electrodes to the surface, and wherein the processor, via the computer program instructions, is configured to output the feedback based on the force being applied by the one or more electrodes to the surface. In some embodiments, the processor outputs the feedback based on a strength of the bioelectrical signals detected by the one or more electrodes.

In some embodiments, the system further comprises an output interface to display the second position of the one or more electrodes and the origin location. In some embodiments, the output interface further comprises an electrode overlay, wherein the origin location and the second position of the one or more electrodes are visualized onto the electrode overlay. In some embodiments, the electrode overlay comprises a predetermined configuration overlay to depict one or more desired electrode placement locations, and wherein the feedback corresponds to the second position of the one or more electrodes relative to the one or more desired electrode placement locations. In some embodiments, the electrode overlay is generated based on a head width of the subject, a head length of the subject, a head curvature of the subject, or a combination thereof.

In some embodiments, the processor, via the computer program instructions, is configured to catalogue the bioelectrical signals detected by the one or more electrodes. In some embodiments, the computer program is further configured to catalogue the second position of the one or more electrodes corresponding to the bioelectrical signals received by the one or more electrodes.

In some embodiments, the processor outputs the feedback based on a strength of the bioelectrical signals detected by the one or more electrodes. In some embodiments, the feedback comprises haptic feedback, visual feedback, or a combination thereof. In some embodiments, the output interface displays the second position of the one or more electrodes. In some embodiments, the output interface further comprises an electrode overlay, wherein the second position of the one or more electrodes are visualized onto the electrode overlay. In some embodiments, the electrode overlay comprises a predetermined configuration overlay to depict one or more desired electrode placement locations, and wherein the feedback corresponds to the second position of the one or more electrodes relative to the one or more desired electrode placement locations. In some embodiments, the electrode overlay is generated based on a head width of the subject, a head length of the subject, a head curvature of the subject, or a combination thereof of a subject.

In some embodiments, the system is configured to enable self-administration of an EEG analysis, ECG analysis, EMG analysis, or a combination thereof. In some embodiments, the system enables the subject to self-administer an EEG analysis, ECG analysis, EMG analysis, or a combination thereof. In some embodiments, the surface is a skin surface of a chest of the subject, and wherein the system enables administration of an ECG analysis. In some embodiments, the surface is a skin surface of a scalp of the subject, and wherein the system enables administration of an EEG analysis. In some embodiments, the surface is a skin surface of the subject, and wherein the system enables administration of an EMG analysis. In some embodiments, the skin surface is located on an arm of the subject. In some embodiments, the system is configured to transmit electrical signals to the surface using the one or more electrodes.

Provided herein are embodiments of a computer implemented method of guiding placement of one or more electrodes on a surface comprising: calibrating an initial position of the one or more electrodes; tracking a deviation of a second position of the one or more electrodes relative to the initial position using one or more sensors; and providing a feedback based on the deviations.

In some embodiments, the one or more sensors comprise an inertial sensor, an image sensor, or a combination thereof. In some embodiments, the image sensor comprises a camera. In some embodiments, the camera comprises a wide-angle, camera, a color camera, an infrared (IR) camera, a depth camera, a 3D camera, or a combination thereof.

In some embodiments, the feedback comprises haptic feedback, visual feedback, or a combination thereof. In some embodiments, the method further comprises a step of displaying a current position of the one or more electrodes. In some embodiments, the current position of the one or more electrodes is displayed on an output interface of a computing device.

In some embodiments, the method further comprises a step of setting an origin on the surface; and determining one or more desired electrode placement locations relative to the origin. In some embodiments, the one or more desired electrode placement locations correspond to a 10-20 electrode placement system. In some embodiments, the method further comprises a step of providing feedback based on a current position of the one or more electrodes relative to the one or more desired placement locations. In some embodiments, the feedback comprises haptic feedback, visual feedback, or a combination thereof. In some embodiments, the method further comprises a step of displaying the current position of the one or more electrodes and the one or more desired placement locations. In some embodiments, the current position of the one or more electrodes is displayed on an output interface of a computing device.

In some embodiments, the step of calibrating the initial position comprises applying an edge detection algorithm via a computing device in operative communication with the one or more sensors. In some embodiments, the method further comprises detecting one or more electrical signals from the surface with the one or more electrodes. In some embodiments, the method further comprises a step of providing feedback based on the strength of the one or more electrical signals. In some embodiments, the one or more electric signals comprise bioelectric signals. In some embodiments, the method further comprises a step of cataloguing the one or more electrical signals. In some embodiments, the step of cataloguing the one or more electrical signals comprises cataloguing a position of at least one electrode of the one or more electrodes which detects the one or more electrical signals. In some embodiments, the step of cataloguing the one or more electrical signals comprises storing the position of the at least one electrode and the corresponding one or more electric signals obtained by the at least one electrode to a data storage medium.

In some embodiments, the one or more sensors comprise an image sensor, and wherein the method further comprises cataloguing images obtained by the image sensor. In some embodiments, the one or more sensors comprise a force sensor, and wherein the method further comprises cataloguing force values measured by force sensor. In some embodiments, the one or more sensors comprise an inertial sensor, and wherein the method further comprises cataloguing rotation and acceleration measured by the inertial sensor.

In some embodiments, the method further comprises a step of transmitting one or more electric signals to the surface with the one or more electrodes. In some embodiments, the method further comprises a step of sensing a force applied to the surface by the one or more electrodes. In some embodiments, the method further comprises a step of providing feedback based on the force applied to the surface by the one or more electrodes. In some embodiments, the force applied to the surface by the one or more electrodes is measured by one or more force sensors.

In some embodiments, the step of detecting deviations from the initial position comprises receiving data from one or more inertial sensors. In some embodiments, the one or more inertial sensors comprise at least one accelerometer. In some embodiments, the one or more inertial sensors further comprise at least one gyroscope.

In some embodiments, the step of detecting deviations from the initial position and the initial orientation comprises receiving data from a camera. In some embodiments, the step of detecting deviations from the initial position and the initial orientation further comprises detecting the origin with the camera and evaluating the position of the origin in a series of images captured by the camera. In some embodiments, the origin comprises an edge detected by an edge detection algorithm used to evaluate the series of images captured by the camera.

In some embodiments, the method enables a subject to self-administer an EEG analysis, ECG analysis, EMG analysis, or a combination thereof. In some embodiments, the surface is a skin surface of a chest of a subject, and wherein the method enables administration of an ECG analysis. In some embodiments, the surface is a skin surface of a scalp of a subject, and wherein the method enables administration of an EEG analysis. In some embodiments, the surface is a skin surface of a subject, and wherein the method enables administration of an EMG analysis. In some embodiments, the skin surface is located on an arm of the subject. In some embodiments, the method further comprises transmitting electric signals to the surface using the one or more electrodes.

In some embodiments, the method further comprises a step of sensing a force applied to the surface by the one or more electrodes. In some embodiments, the method further comprises a step of providing feedback based on the force applied to the surface by the one or more electrodes. In some embodiments, the force applied to the surface by the one or more electrodes is measured by one or more force sensors. In some embodiments, the one or more force sensors comprise a piezoelectric transducer.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a rapid positioning system configured for electroencephalogram (EEG) acquisition or electro-stimulation from the subject's scalp, according to some embodiments.

FIG. 1B depicts rapid positioning system on a subject's arm for electromyography (EMG) recording or muscle group stimulation, according to some embodiments.

FIG. 1C depicts a subject's chest with a rapid positioning system for electrocardiogram (ECG) acquisition or electro-stimulation, according to some embodiments.

FIG. 1D depicts a rapid positioning system configured as a barrel or housing with mechanical extensions for the electro-stimulation of a subject's lower back, according to some embodiments.

FIG. 1E depicts a rapid positioning system configured as a barrel or housing with mechanical extensions for EEG acquisition at multiple configurations on the subject's scalp during one session, according to some embodiments.

FIG. 2 is a block diagram illustrating systems and instruments in a rapid positioning system for tracking, acquiring or electro-stimulating, in accordance with some embodiments of the invention.

FIG. 3A is a flow chart depicting a method of implementing a rapid positioning bioelectrical signal acquisition and stimulation system, according to some embodiments.

FIG. 3B is a flow chart depicting methods of implementing a rapid positioning system at the beginning of a session, according to some embodiments.

FIG. 3C is a flow chart depicting methods for validating the electrode configuration placement for a rapid positioning system, according to some embodiments.

FIG. 3D is a flow chart depicting methods for cataloging and managing process related data from a rapid positioning system, according to some embodiments.

FIG. 4A depicts a user interface on an output tablet for EEG acquisition, according to some embodiments.

FIG. 4B depicts visual cues on a user interface to display electrode configuration, placement validation results, and the quality of the connection with the subject's scalp to enable reproducibility, according to some embodiments.

FIG. 5 depicts data cataloging and storage of EEG signals recorded from multiple unique configurations, according to some embodiments.

FIG. 6 depicts an integrated computer system, according to some embodiments.

### DETAILED DESCRIPTION

Measuring bioelectrical signals from living subjects and providing electrical stimulation to living subjects may be used in several diagnostic and treatment contexts. For instance, electrodes may be arranged on the skin to record bioelectrical signals from the heart, known as the electrocardiogram (EKG or ECG) that provide information about the functioning of the heart. Cardiologists may utilize the ECG to detect abnormal or irregular heart rhythms, or to detect ischemia such as during a heart attack. Similarly, electrodes may be arranged in different configurations on the scalp of a subject to record bioelectrical signals from the brain, known as electroencephalogram or EEG. EEG signals may be used to assess brain activities including cognition, seizures, psychogenic illnesses, effects of anesthetics, and sleep disorders. Electrode configurations can also be placed over muscle groups located in arms, chest, legs, or other areas of interest to record a class of bioelectric signals known as electromyogram or EMG that are generated by the contraction of muscle groups. EMG signals may be used for diagnosis of various conditions and have been utilized to operate artificial prosthesis such as hands, arms, or lower limbs as well as other brain computer interfaces.

Therapeutic uses of electrical stimulation delivered to certain brain regions may include the treatment of Parkinson's disease, bulimia, epilepsy, and other neurological disorders. Likewise, electrical stimulation to the heart, such as through defibrillators and pacemakers, may be used to restore normal heart rhythm. Electrical stimulation delivered to parts of the nervous system (referred to as neuromodulation) may be used to alleviate pain or control certain body activities. The acquisition and stimulation of electrical signals from or to a living subject may form the underlying basis for several medical diagnoses and treatments.

The process of acquisition or stimulation may be achieved by attaching electrodes in a determined configuration on a body. The skin may be exfoliated to remove dead skin cells, oil, and other impurities prior to placing electrodes to improve the quality of signal by reducing the electrode-skin impedance. An example of a single electrode is an ECG electrode, which typically uses a silver coated electrode, pre-impregnated with gel containing silver chloride, a means to attach a wire (lead) to the electrode and surrounded by an adhesive collar to attach to the skin. Existing electrode positioning systems utilize a large number of electrodes to ensure reliable and reproduceable signals are measured. However, these existing systems are complex, time consuming, and may hinder use for short-term screening or urgent electrical stimulation treatment because they require a trained clinician to operate.

Wearable designs are available that may use tape, glue, bands, straps, caps or headsets to mechanically supply force and secure the electrodes to the determined configurations on the scalp. Existing wearable designs confine the electrode locations to a specific configuration which provides value for long-term monitoring. However, confinement results in a long and complex setup which inhibits the use of these designs for short-term screening or urgent electrical stimulation treatment.

Attempts have been undertaken to create confined wearable systems with limited number of electrodes that may reduce the complexity of setup. However, they present their own set of challenges. For example, an inflatable wearable headset EEG system may cover a wide range of sites on the subject's scalp but may not be applicable for rapid deployment during clinical emergencies. Such inflatable headset systems may increase the set-up challenges for a medical practitioner as their relatively rigid geometry may not accommodate all head shapes and sizes. Such headsets may also inconvenience the medical practitioners, as they may be required to completely remove the headset to exfoliate scalp sites with poor signal quality, an important step for improving the skin-contact and reducing background noise. In another example, an EEG headband with limited number of recording electrodes that is fixated around the crown of a subject's head may lack coverage of all areas of interest especially the center of the subject scalp, limiting their monitoring applications to specific brain conditions only. Other wearable EEG headset recording systems, such as those with 5 electrodes, suffer from mechanical restrictions imposed by the headset design that limit the applications of the system to specific use cases only.

Existing wearable EEG recording systems with an array of sensing electrodes in a fixed pattern worn on at least one finger (or that can be worn like a glove) have multiple loose wires that can tangle with the operator while performing the recording. Previously described wearable EEG recording systems configured like a pen lack a reliable and reproducible positioning system and rely solely on the skill of the trained technician. Without a reliable position tracking the user may be required to manually estimate and note the location of each electrode applied on the subject based as well as accurately match the EEG data to the location on their recollection of the administering the recording session. Such operational workload may not only hamper the ease-of-use but may also create discrepancies in post-hoc data storage for interpretation and diagnosis. Such reproducibility in configuration placement and accuracy in post-hoc storage is especially important, as it is critical to maintain structured and consistent inputs for training a Machine Learning model for future advancements.

Existing positioning systems for wearable EEG devices may not be accurate, at least because they fail to automate the device configuration detection on the subject's body and because they lack the resolution to identify the unique subject's habitus to precisely locate the placement of each sensor. In addition, existing positioning systems may lack the spatial resolution to discern unique configurations at short distances on the subject's body, such as the scalp or certain muscle groups. This limitation may severely hamper the collection of meaningful EEG signals as, unlike ECG, EEG electrode placement may lack palpable landmarks, and may require an expert to locate the 10-20 individual sites of the system on the subject's scalp. Improper positioning of the EEG electrodes may result in erroneous data cataloging as there may be unique signatures recorded from individual 10-20 sites that may be absent in other sites.

There is a growing need in remote collection and analysis of signals for use in telemedicine. Such systems may require the accurate identification of the device placement while providing users the feedback to ensure reproducibility and reduce discrepancies during data storage and interpretation. Existing systems and methods for remote evaluation of sensor data acquired from the heart and lungs of a patient are not suitable for EEG, due to design challenges. LED and camera-based system that track the placement of the device present several limitations as a patient will be required to sit still in a particular orientation to ensure the subject outline is captured in the frame and the camera calibration is successful. Such techniques may not be applicable if the patient is unconscious and a medical practitioner with limited training has to administer the recording. Additional limitations include the inconvenience that may be caused during calibration for the collection of EEG signals where a patient may be lying flat on the hospital bed and it is challenging to obtain correct camera positioning.

Quality of the EEG signal may be paramount in performing a diagnosis, and signals that are corrupted by noise, artifacts (e.g.: oculomotor electrical signals), or suffer from poor electrode-skin interface may not be useful, especially for remote monitoring where the expert may not be present on-site. Automated quality assessment of EEG for brain-computer interface systems (BCI) have been may require computing resources available for BCI systems that are not available for a rapid handheld diagnostic test to be used, for example, in an emergency situation.

There exists a need for an electrode tracking system to accurately automate the configuration detection with high spatial resolution for handheld screening tools and ensure reliability and reproducibility in device placement amongst users with different types and levels of expertise. Despite all attempts to solve the clinical problem of diagnosing suspected neurological conditions with EEG in an emergency, there is still no device that is simple, easy to use, portable (e.g.: carried in a pocket), capable of providing high quality monitoring, and complete with apparatus and method of reliable positioning able to be implemented by a non-expert user. Such ease-of-use may also allow non-expert users to reliably self-administer the device from the comfort of their home.

In the following sections, references to embodiments are provided to build context and thorough understanding of the invention. However, the invention is not limited to the mentioned embodiments and can be extended to other embodiments that lack design-imposed electrode confinement to the subject's body and can be deployed as rapid positioning systems. For example, the methods and systems described herein may be useful for acquiring EEG signals from the brain as well as other biosignals, such as ECG signals and EMG signals.

### I. HANDHELD SYSTEMS

Provided herein, in some embodiments, are systems, devices and methods to utilize different handheld systems to acquire signals or electro-stimulate regions on the subject's body. In some embodiments, the handheld systems comprise one or more electrodes for measuring electric biosignal or for providing electrical stimulation. The tracking system may track and record the positioning of the electrodes relative to the subject on which they are placed. In some embodiments, positioning of the electrodes is tracked relative to a set origin or landmark on a subject, as disclosed herein.

In some embodiments, tracking of the electrodes enables proper positioning of the electrodes at one or more sites. In some embodiments, the sites at which the electrodes are placed are based on the standard 10-20 electrode placement system for obtaining biosignals from a scalp of a subject. The sites may include the sites of F3-F4-Cz, T3-T5-M1 and O2-T6-M2 based on the standard 10-20 electrode system. The tracking system may allow placement of the electrodes on to the sites with little training or pre-existing knowledge of the standard electrode site locations.

In some embodiments, the tracking system provides coordination of bioelectrical signals with the sites at which they were recorded. This data may be catalogued, with the signals corresponding to the locations at which they were obtained. This may reduce additional tools or systems require to correlate signals obtained with the placement of the electrodes. In some embodiments, wherein the electrodes provide electrostimulation to one or more sites, the tracking system may prevent repeated stimulation at a site, when not desired. For instance, if one site is to only be treated with an electrostimulation signal once, the tracking system may prevent the device from providing additional electrostimulation signals to the site.

A. Device with Housing

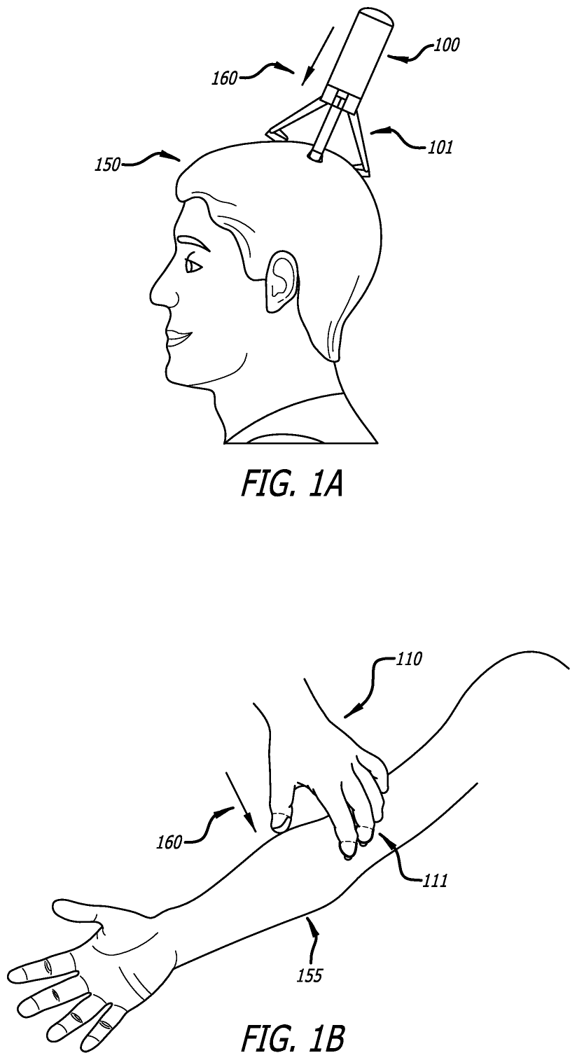

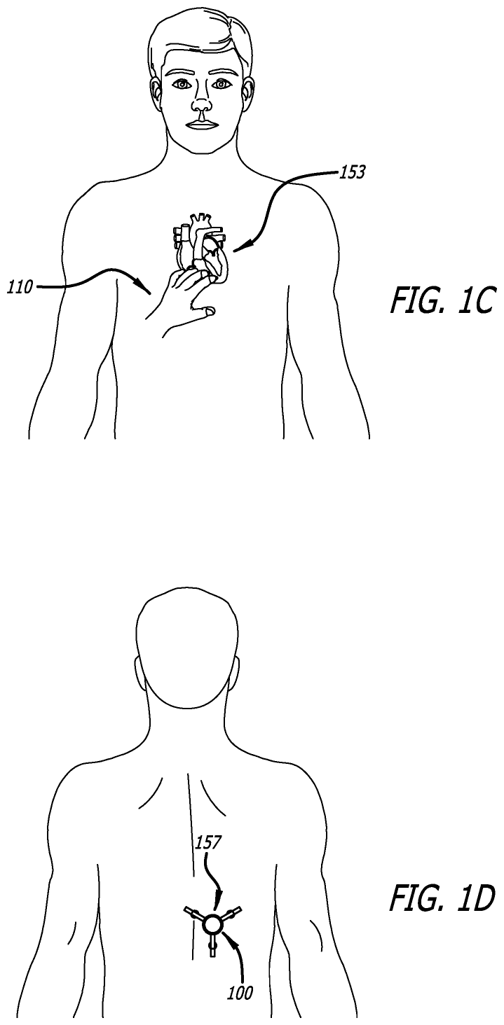

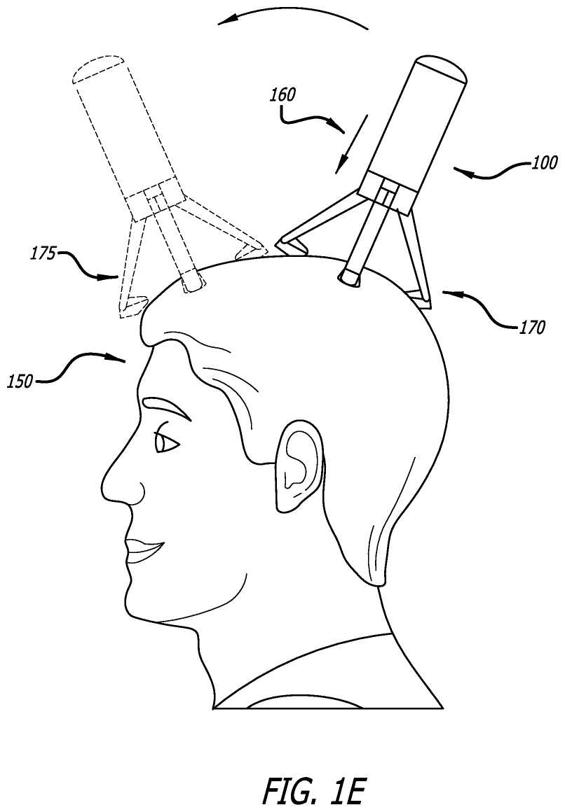

Referring to FIG. 1A, depicts a device 100 that can be positioned on the scalp 150 of the subject for bioelectric signal acquisition or stimulation is provided, according to some embodiments. In some embodiments, the device 100 has extensions 101 to carry electrodes at the distal end in a specific configuration on the subject scalp 150 to achieve an action. Configurations created by such devices 100 may depend on the organization of the extensions 101 and the inter-extension distance which in some cases may be physically altered through automated or manual techniques to create different configurations. In some embodiments, the device is temporarily placed on the subject scalp 150 by a downward user-applied force 160 to achieve an action. Similarly, the devices may be withdrawn from a configuration on the scalp 150 by the removal of the applied force for repositioning on another configuration. In some embodiments, the downward user-applied force 160 on the electrode for connection with the subject's scalp 150 may be further improved by the application of conductive gel. In some embodiments, the electrodes are connected by wires that run along extensions 101 to a central barrel unit that contains necessary electronics to store and transmit the acquired data.

B. Glove with Finger Caps

FIG. 1B depicts of a finger-wearable device 110 for bioelectric signal acquisition or electro-stimulation that can be placed at a specific configuration on the subject's arm 155 to execute an action on a muscle group, according to some embodiments. Configurations for such devices 110 may be formed by varying the distance between the finger caps 111 that house the electrodes. Similar to device 100 in FIG. 1A, the finger-wearable device 110 may be positioned on the arm 155 by a downward user-applied force 160 at the finger caps 111 to accomplish an action and repositioned to another configuration by removing the force. Such devices may carry any necessary electrode assembly and components at the finger caps 111 and have wire connections run to a central unit for device management. In some embodiments, the central unit may either be worn around the wrist by the user and may contain electrode connectors that can attach to the electrodes prior to device use.

FIG. 1C depicts another placement of the device 110 in a specific configuration on the subject's chest 153 to collect bioelectrical signals or electro-stimulate the heart according to various embodiments described herein. In some embodiments, the placement as shown in FIG. 1D of the device 100 is provided for acquiring or electro-stimulating a subject's lower back 157 region.

FIG. 1E depicts an implementation of a device 100 to record EEG signals from the subject's scalp 150 at multiple configurations 170, 175, according to some embodiments. Device 100 lacks design restrictions that confine electrode positions at a first configuration 170 and may be repositioned to greater than one or more configurations 175 by applying downward force 160 or releasing it. Such repositioning enables the user to gain more coverage and capture unique brain signatures on the scalp 150 during EEG acquisition with fewer electrodes.

Such devices 100 and 110 may include additional tracking instruments to cataloged EEG information collected from multiple configurations for accurate storage. For instance, three electrodes may be sequentially deployed on the subject's scalp for short periods of time to monitor multiple configurations such as F3-F4-Cz, T3-T5-M1 and O2-T6-M2 based on the standard 10-20 electrode system. In some embodiments, the user utilizes three electrodes to monitor the brain activity from nine unique sites on the subject's scalp and the tracking instruments would reliably catalog the data of each electrode to the associated configuration.

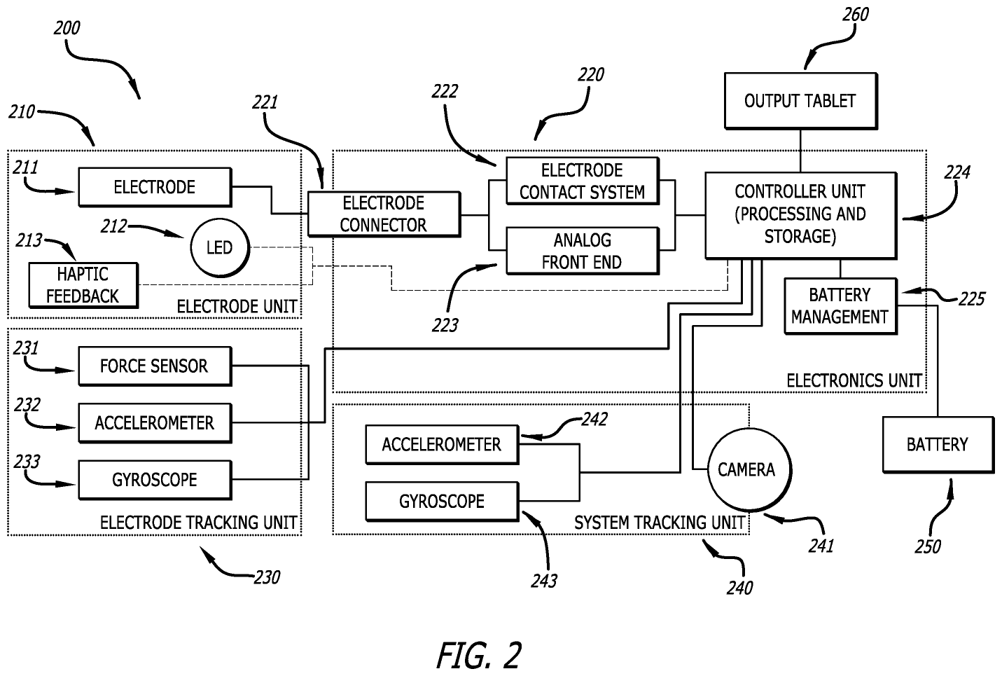

FIG. 2 provides a schematic 200 of systems and instruments enclosed in the rapid positioning device 100 and 110 to enable its functioning according to some embodiments described herein. In some embodiments, the electrode unit 210 interfaces with electronics unit 220 for sensing one or more signals obtained from a living subject or delivering an electrical stimulation. The electrode unit 210 may include one or more biopotential electrodes 211. Such electrodes 211 may be wet, semi-dry, dry, or active. The disclosed inventive concepts may employ either homogeneous or heterogeneous combination of aforementioned electrode types to accomplish the purpose.

In some embodiments, an electrode 211 is attached to the distal ends of the device 100 at 101 for convenient access to the subject's skin or scalp. In some embodiments, the electrodes 211 for finger caps 110 are attached to the base of the cap at 111 or any similar position to obtain contact with subject's skin or scalp. The electrode 211 may connect through an electrode connector 221 that passes the information to an analog front-end CMOS chip 223 which is capable of filtering, amplifying and digitizing the signal. In some embodiments the analog front-end 223 may comprise individual electrical components, such as resistors, capacitors, amplifiers, etc., for analog signal acquisition. In some embodiments, the digital output from the analog front-end 223 gets buffered in the controller unit 224 prior to either a wired or wireless (Bluetooth or RF) transmission to an output tablet 260. In some embodiments, the buffered data in the controller unit 223 may be wirelessly transmitted to a cloud storage for further processing and visualization on a web-based application.

The controller unit 224 may be divided into more than one controller units which may include network modules and memory storage. In some embodiments, an embedded system software may be loaded on the memory storage that can be accessed by the controller unit 224 to act as the operating system of the device. This operating system may perform various actions to interface with the analog-front end for communicating with the electrodes, managing wired or wireless data transfer to an output tablet 260 or directly to a cloud storage that can be accessed by a web-based application, handling service requests created by the user on the output tablet 260, performing low-level signal processing or managing instruments in electrode tracking unit 230 and system tracking unit 240. Such controller units 224 may be adequately powered by a battery source 250 through a battery management system 225. Battery management systems 225 may perform various functions including voltage regulation, recharge of the battery through an external power source and distribution of adequate power supply to any electrical components as per the requirements. In some embodiments, the battery management system 225 may be programmed to collect information about the battery level or its performance. In some embodiments, the battery management system 225 may be programmed to operate in low-power mode or increase the power supply when the user-interface is activated.

Some of the embodiments may contain an electrode contact system 222 in the electronics unit 220 comprised of necessary electronics to electrode impedance an electric current at a specified frequency to two or more electrodes 211. In some embodiments, the electrode contact system 222 can also be configured to function as an electro-stimulator by delivering varying levels of user-defined current to the subject's body.

### II. ELECTRODE TRACKING

In some embodiments, aspects of the present disclosure provide methodologies specific to rapid positioning systems that lack design-imposed electrode placement restrictions and can be positioned by applying a short-term user-driven/user-applied force at determined configurations on the subject's skin or scalp. Such rapid positioning systems may be easily detached from the initial configuration by releasing the user-applied force and repositioned to another configuration on the subject's skin or scalp, temporarily fixing the electrode by reproducing the user-applied force. In some embodiments, this repositioning technique enables the disclosed systems to cover multiple configurations on the subject's skin or scalp with fewer electrodes with high reliability and reproducibility in comparison to the commercially available fixed-electrode or portable bioelectric signal system counterparts. Repositioning techniques in the present disclosure may also cover minor adjustments required to either achieve optimal electrode-skin contact or a precise area of interest.

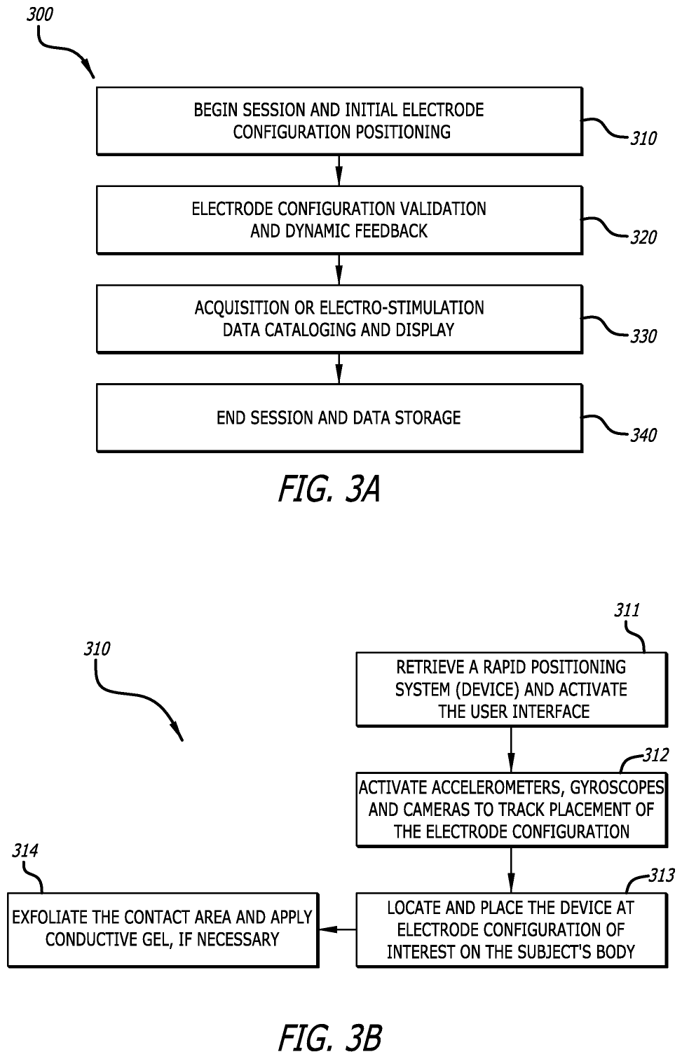

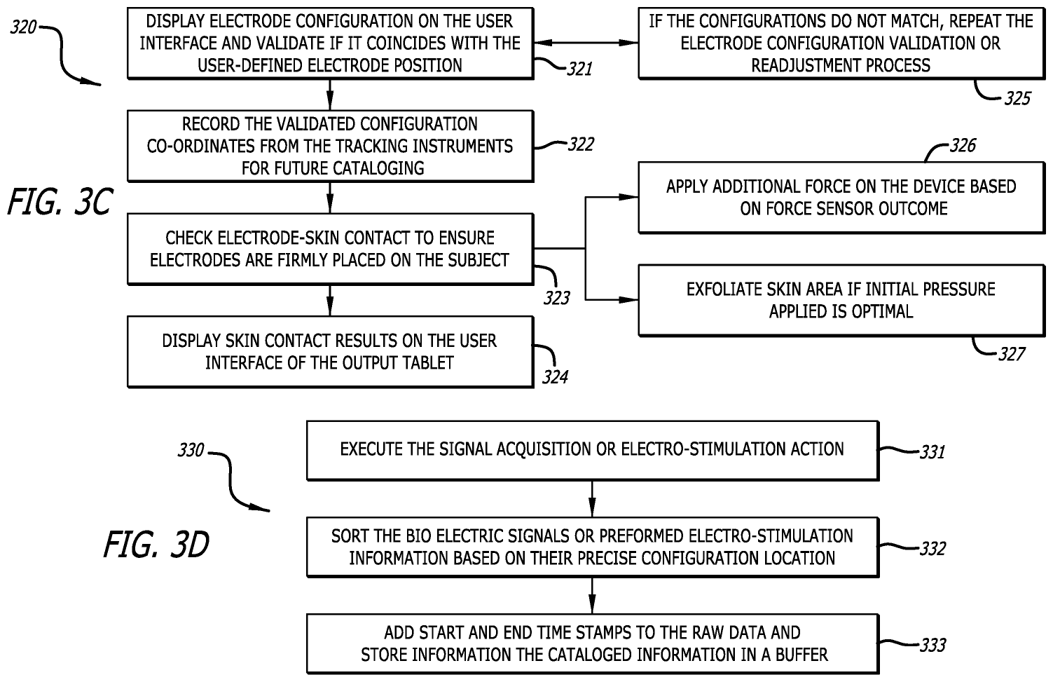

FIG. 3A depicts a process flowchart 300 of utilizing the device 100 or 110 to perform a bioelectric signal acquisition or stimulation action, according to some embodiments. Such devices may carry a minimum of one electrode to execute the action and the number of determined configurations (A-N) to be monitored or stimulated based on the user's discretion. At step 310, a user may initiate a bioelectric acquisition session or electrical stimulation session. In some embodiments, a session may be initiated by a user input to calibrate the positional sensors (e.g., inertial sensor as described herein) and/or image sensors (e.g., a camera as described herein) of the system. In some embodiments, initiation of the system comprises setting an origin or reference point from which displacement of the one or more electrodes will be tracked (e.g., via one or more inertial sensors and/or one or more image sensors). In some implementations, the device can be utilized to record multiple bioelectric signals or perform both acquisition and stimulation in one session. In some embodiments, the critical element of implementing the rapid positioning technique is to continuously monitor the placement of the electrode configurations on the subject's body. In some embodiments, at step 320, the system provides validation and dynamic feedback to ensure proper placement and contact of the one or more electrodes on a subject. By tracking the device's placement through instruments in the electrode tracking unit 230 and system tracking unit 240, a low-level machine learning algorithm may identify different physical features on the subject (e.g. head, chest, arm or back) and accurately categorize and catalogue the biosignals while maintaining high spatial resolution 330.

In some embodiments, the user may initially position the device on the arm to record EMG followed by placing the device on the chest to record ECG. In both cases, the tracking instruments may be utilized to identify the subject's body parts such as arm and chest to catalog the signals in the correct category. Such automated cataloging techniques may reduce the operational workload for the user while providing them the freedom to efficiently monitor a number of configurations. Tracking may also be implemented for EEG signals to identify both the short-range (intra-lobe) and long-range (inter-lobe) configuration changes of the electrode units. Accurately identifying the configurations changes within the 10-20 electrode subset may not only reduce the erroneous cataloging caused by manual input but also provide physicians the necessary coordinate information to interpret and associate the unique biosignal information with the specific brain region. For example, the high frequency oscillations generated from the frontal region of the brain may differ from the high frequency oscillations in the occipital region, making it critical to accurately record the position coordinates of the configuration for physicians to interpret the data after the end of a session 340.

In some embodiments, FIG. 3C depicts further details of the validation process, of step 320, for the configuration placement that may begin after the electrodes are placed firmly against the subject's skin. The information combined from the tracking instruments 230 and 240 (e.g., one or more inertial sensors, one or more image sensors, and/or one or more force sensors) may be utilized to calculate the spatial 3D coordinates of the device. The estimated position of the device may be calculated by measuring the difference between the 3D position coordinates when the tracking unit calibration process was initiated and when the force sensor 231 detects that the device was placed on the subject's skin. The estimated 3D coordinates of the electrode configuration may be translated into an intuitive location tag and marked on a line body diagram displayed on the output tablet. The user may be prompted on the output tablet to validate if the displayed location tag for the configuration coincides with their positioning on the subject's body 321. The validation input may be obtained either through pressing a mechanical button, clicking an option on the output tablet screen, or voice input collected through a microphone in the output tablet. If the user validates the configuration, the location tag may dynamically adapt as a visual cue to reflect the successful validation. The estimated 3D coordinate information may be recorded in a buffer for future use 322. There may be cases when there is a misalignment between the actual placement of configuration on the subject compared to the location tag displayed on the output tablet. In such instances, the user can either manually choose the configuration on the output tablet or restart the tracking process 325. At step 323, the output tablet may initiate a service to test for the quality of the electrode-skin contact to ensure that the user has firmly placed the electrodes against the subject's skin. For example, one or more force sensors as described herein may be used to ensure the electrodes are firmly placed against the subject's skin. The user may receive a feedback to adjust the force of the electrodes applied to the subject, as detected by the one or more force sensors (step 326).

Accordingly, the one or more inertial sensors, the one or more image sensors, and/or the one or more force sensors may be used singularly or in combination to help enable reliable and reproducible signals are acquired from the subject via the electrodes. The use of such inertial sensors, image sensors, and/or force sensors help guide the placement (positionally and/or with respect to skin contact) of the electrodes, such that a user, subject, and/or other operator with limited training will be able to acquire said reliable and reproducible signals from subject. As used herein, the term subject may refer to a person (such as a patient) on whom the electrodes are being placed. As used herein, the term user and/or operator may refer to a person who is operating a device described herein. In some instances, the subject and the user are the same, wherein the subject operates a device described herein on themselves.

In some embodiments, FIG. 3D depicts further details the acquisition or electro-stimulation action may be initiated at step 330. The output tablet may collect user-input to conduct an acquisition or a stimulation session. The input action may be processed and executed 331. In some embodiments, the user input may be collected through buttons available on the output tablet interface. For example, the user input may be collected through a physical switch available on the device or through voice commands recorded by microphones on the output tablet 260. The user input may be interpreted or processed to make necessary adjustments on the output tablet interface to display the validated configurations, critical metrics like the skin contact quality, noise analysis, and other quantifiable parameters. At step 332, the acquired biosignal (e.g., EEG, EMG or ECG) or performed electro-stimulation information (e.g., amplitude, phase, frequency or duration) may be associated with the configuration coordinates recorded at step 322. Time stamps of the action initiation and conclusion may also be recorded as additional information for data cataloging and storage purposes 333. The recorded timestamps may originate from the output tablet's or the controller unit's internal clock. All recorded information during an active session can be held at a temporary location before moving to a permanent storage. The user may be interested in more than one configuration. In some embodiments, the user can repeat steps 310-330 with associated sub-steps for another user-selected configuration. At step 340, the user may end the session and the data can be moved to a permanent location that is either on the device hardware, output tablet, a removable SD card, online data storage reserves or any other data storage methods. In some embodiments, the validation step 321 can be accomplished after the signal acquisition or electro-stimulation to retrospectively assign the correct electrode configuration location for cataloging.

Placement of electrodes often requires medical practitioners to undergo extensive training. Training needs to be broad enough to be applicable to different health conditions, especially for EEG but specific enough to prepare the practitioner for use of the device. The training may be enhanced by suggesting specific predetermined electrode configurations based on compiled clinical research to screen or treat a subject. In other instances, medical practitioners may be guided to predetermined electrode configurations for a specific ailment where an electro-stimulation treatment might be beneficial. Such techniques may be paired with remote access through telemedicine, where a trained and experienced medical practitioner can instruct the untrained staff to place the device at predetermined configurations based on the symptoms exhibited by the subject. The collected data may be sent to the experienced medical practitioner to assist in making a clinical diagnosis. In some instances, the experienced medical practitioner may supervise the process 300 administered by the untrained staff in real-time through telemedicine. In some instances, the output tablet can perform necessary computations to determine the outcome from the screening process.

In some embodiments, the system allows the practitioner to specify a suspected diagnosis and receive instructions to position the device at specific predetermined locations to enable rapid screening process. At the beginning of the session 310, the user may have an option to input a specific biosignal/electro-stimulation of interest and subsequently an ailment or region of interest on the output tablet once the user interface is activated 311. With the provided user input, the interface of the output tablet may be modified to display the electrode configurations that are to be monitored or stimulated. The user can review the displayed configurations 480, locate them and place the device on the subject's body 313. The tracking instrument activation and calibration may be carried out as detailed in step 312 and, if necessary, exfoliation of subject skin may be performed 314. The sub-step 321 may be modified to perform an automated validation of the electrode configuration placement by confirming if the estimated 3D coordinates of the positioned device coincide with the suggested configuration. The system may allow a small but acceptable margin of misalignment as most biosignals that originate from neurons spread radially at the surface of the skin. In some embodiments, the automated process can be overridden by manual input at user's discretion. In situations where the estimated coordinates do not coincide with the suggested configuration, the output interface may prompt targeted suggestions for readjustment. Once the coordinate information is validated, the result is displayed on the output tablet and the coordinate information is recorded for future data cataloging 321. At the end of step 330, the output tablet may propose additional configurations to monitor or electro-stimulate based on compiled clinical research or the experienced medical practitioner's request. In some embodiments, the user can repeat steps 310-330 with associated sub steps including the variations proposed when the suspected diagnosis is specified.

A. Positional Tracking

Inertial sensors such as accelerometers 232 and gyroscopes 233 may also be enclosed in the electrode tracking unit 230 to record the position of the electrode unit 210 in real-time. The information from the force sensor 231, electrode contact system 222, accelerometer 232 and gyroscope 233 is transferred to the controller unit 224 for additional processing and interpretation. A system tracking unit 240 containing inertial sensors such as accelerometers 242 and gyroscopes 243 or an image sensor (e.g., camera 241) may be included in some embodiments to act as a reference for monitoring the electrode unit 210 position with respect to the subject's body. A camera 241 in the system tracking unit 240 may a wide-angle, camera, a color camera, an infrared (IR) camera, a depth camera, a 3D camera, or a combination thereof. Some system tracking units 240 may utilize inertial tracking mechanisms by combining inertial information from accelerometers and gyroscopes located in the electrode tracking unit 230 and system tracking unit 240.

For example, the user may pick a desired landmark on the subject body and spatially calibrate the device to set a location origin. Any inertial changes with reference to the origin may assist in the 3D subject visualization. Some tracking units may combine the inertial information from the electrode tracking unit 230 and system tracking unit 240 with the visual input obtained from a camera system 241 for 3D subject visualization. Such tracking techniques may utilize a frame-based feature tracking through a low-level machine learning algorithm. In some embodiments, images from a color camera system can are processed to extract edges of critical visual features and identify the subject's body outline. The positional changes in the critical visual features will be combined with the inertial data obtained from the sensors 232, 233, 242 and 243 to compute the change in position. IR and depth camera tracking techniques may follow similar edge detection and visual feature tracking methodology to detect changes in position. Integration of configuration detection using image and inertial information also enables the identification of unique subject habitus allowing for customized and accurate determination of the placed configuration on both adults and infants alike. The tracking information will either be processed locally on the device hardware or wirelessly transmitted to an output tablet 260 with sufficient computational power for processing and yielding meaningful visual cues and alerts for the user. In some embodiments, the placement of the aforementioned electronics, instruments and systems in different electrode unit 210, electronics unit 220, electrode tracking unit 230, and system tracking unit 240 was undertaken to enhance the understanding of the art. The instruments and electronics are not required to be physically confined to their categorized unit. The aforementioned instruments, electronics and systems may be added, removed or altered in their physical positions on the device without sacrificing the performance and reproducibility of the rapid positioning system.

### III. SYSTEM FEEDBACK

In some embodiments, one or more modes of feedback are provided to a user during use of the systems. The feedback system may contain either an LED 212, haptic feedback 213 or a combination of both instruments.

A. Visual Feedback

In some embodiments, the light emitting diode (LED) 212 attached to the electrode unit 210 delivers visual feedback to the users regarding the electrode contact or correct/incorrect positioning. The LED 212 may signify a specific state of the device by using various colors such as red, orange or green. In other implementations, the LED 212 may also utilize a blink feature to communicate the device state.

B. Haptic Feedback

In some embodiments, a small vibration generator will be included to provide haptic feedback 213 about the precision of the device position on the subject's body and other metrics. For example, the frequency or amplitude of vibration can change with proximity to the best position to guide the user. In some embodiments, haptic feedback is utilized to indicate a state or activation of the device.

C. Output Interface

FIG. 3B outlines the process for the user to begin the acquisition or stimulation session 310 by retrieving the rapid positioning system (device) while activating the user interface 311 on the accompanying output tablet 260 for pairing and viewing information, according to some embodiments. In some embodiments, the device may pair instantly through wireless telemetry such as Bluetooth. In some embodiments, a wired connection may be established through a cable between the device and the output tablet to execute the process. In some embodiments, the device may wirelessly send information through network technologies to cloud-storage which may be accessed by an output interface on a web-based application. In some embodiments, instruments in the electrode tracking unit 230 and the system tracking unit 240 are activated to monitor the electrode configuration placement 312 with respect to the subject's body. The result derived from the positional information may be displayed on the output interface to the users to provide an intuitive medium to validate the accuracy of the positioning. When activated, such tracking instruments may undergo necessary calibration to ensure accurate results. Tracking implementations that utilize both the visual and inertial inputs may provide the convenience to calibrate the system irrespective of the subject's orientation (for e.g.: standing, sitting or sleeping). In some embodiments, a camera that is attached to the device moves along with the system thus increasing the chances of capturing the subject in the frame. Once the user identifies the electrode configuration of interest, they may locate the positions and place the device on the subject's skin 313. The contact area of the electrode configurations may need exfoliation or application of conductive gel 314 depending on the type of electrodes 211 deployed for improving the performance of the device. The present disclosure contemplates that many variations on the embodiments described herein are possible without changing the intent. For example, the Bluetooth communication could be Wi-Fi, and the tracking camera could use IR camera or depth camera.

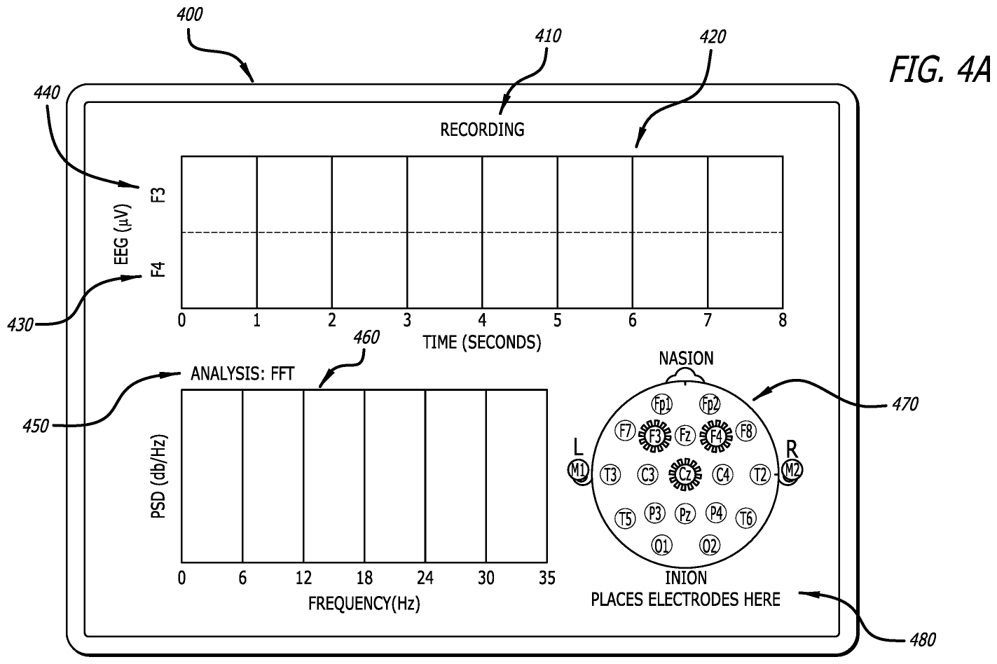

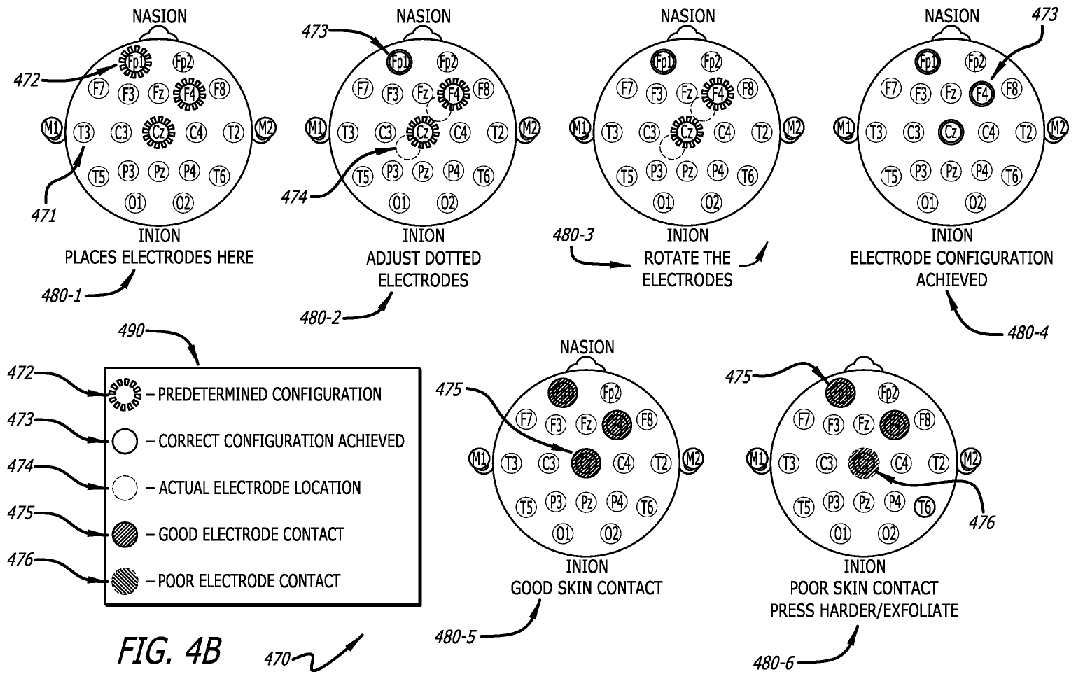

FIG. 4A depicts an exemplary interface 400 displayed on the output tablet 260 that may connect to the rapid positioning system 100 or 110 to record, review and store information. Indication of bioelectric signal acquisition or execution of electro-stimulation session may be highlighted as a label 410 on the screen to aid the user. In instances when the user desires to perform signal acquisition, a label identifying the biosignal of interest 430 and a real-time charting display 420 to view the signals may be available. Such real-time charts may be segmented to display recordings from more than one electrode 211, and each segment may be appropriately marked with the configuration information 440 gathered and validated by the tracking units 230 and 240 at step 322. Additional quantitative metrics for signal analysis may be provided to the user for enhanced use of the system. In some implementations, the quantitative metrics can include real-time fast Fourier transform, root mean square, spectral analysis, coherence between recording electrodes 211 and other signal processing metrics. Such quantitative metrics can be selected based on user-input 460 and may be displayed on a separate real-time chart 460.

In some embodiments, bioelectric signals are acquired for a predetermined duration of time for each configuration. In some embodiments, bioelectric signals are acquired for about 1 second to about 60 seconds. In some embodiments, bioelectric signals are acquired for about 1 second, about 10 seconds, about 20 seconds, about 30 seconds, about 40 seconds, about 50 seconds, or about 60 seconds. In some embodiments, bioelectric signals are acquired for at least about 1 second, about 10 seconds, about 20 seconds, about 30 seconds, about 40 seconds, about 50 seconds, or about 60 seconds, including increments therein. In some embodiments, variations of the bioelectric signals acquired over the predetermined duration of time are catalogued.

Visual cues based on outputs from tracking units, force sensors and electrode-impedance information may be separately displayed on an illustration 470, displaying the subject's region of interest where the rapid positioning system may be displayed. The interactive illustration 470 may adapt to the changes in configurations and conditions of the rapid positioning system and may be accompanied by textual feedback 480. In electro-stimulation embodiments, the visual cue illustration 470 may transform to reflect the area of interest on the subject's body. Although FIG. 4A shows an exemplary interface for the EEG data acquisition, other interfaces may be used for recording other bioelectrical signals and performing electro-stimulation. In instances where a user-desires to perform electro-stimulation, the output interface may adapt to show necessary information pertaining to the process. The label 410 may be modified to “Stimulating” and the output tablet may collect user input for critical parameters such as amplitude, frequency, pulse width and duration of the stimulation for each deployed electrode. Such critical parameter inputs may be collected on the output tablet by requesting the user to type the value using a keyboard or select a value on the sliding scale or dropdown menu. Additional user elements such as start and stop session buttons, device battery level indicator etc. may also be displayed on the output tablet interface 400.

1. Electrode Overlay

FIG. 4B, depicts exemplary visual cue illustrations 470 of a 10-20 electrode placement guide that may be displayed on the output tablet 260 while collecting EEG signals at predetermined configurations when a suspected diagnosis is inputted at the beginning of the session. At the beginning of a recording session, the illustration 470 may present a predetermined configuration overlay 472 where the user should place the electrodes 211. Predetermined configuration overlays 472 may be highlighted using different designs or color features that can effectively alert an unsuspecting user. Less relevant configurations 471 may appear less prominent to ensure the user focuses on specific configurations of interest. Intuitive text feedback 480-1 may also be provided to reinforce the visual cue. Predetermined electrode configuration overlay 472 and text feedback 480-1 may be modified to reflect the status of the rapid positioning system in real-time. In some embodiments, coordinates of the device calculated by the tracking units 230 and 240 may be reflected on the illustration 470 in real-time through location tags or actual electrode location overlay 474. For instance, the illustration 470 may begin displaying location tags or actual electrode location overlay 474 starting step 320 when the tracking unit instruments are activated, and the device is placed on the subject's body. A correct configuration overlay 473 may be represented by a unique design or color feature when an actual electrode location coincides with a part of the predetermined configuration. In cases where the complete electrode configuration is not correctly achieved, text feedback may propose the user to make adjustments 480-2. In some embodiments, targeted textual feedback 480-3 may be provided to simplify user's understanding about required adjustments. In instances when the entire electrode configuration is achieved, the predetermined electrode configuration overlay 472 may be modified to a correct configuration achieved overlay 473 to reflect the change alongside with accompanying text feedback 480-4. The predetermined configurations' estimated positions may be custom-tailored to an individual's head based on the American Clinical Neurophysiology Society guidelines. Electrode configuration co-ordinates for the 10-20 placement may be generated for a range of head lengths and widths for adults and children across different genders. The predetermined configurations' estimated positions may be recommended based on the head length and width estimated during the calibration process. In some embodiments, an electrode overlay visual is generated based on a patient's head shape and size. In some embodiments, the length, width, and/or curvature of the patient's head are utilized as inputs to generate a custom electrode overlay or map.

In some embodiments, feedback from the electrode-skin contact results calculated at step 323 and 324 may be displayed on the output interface illustration 470. Electrodes with sufficient contact and impedance for optimal performance may be displayed as good electrode contact overlays 475. Alternatively, electrodes that require additional preparation or adjustments may be highlighted as poor electrode contact overlay 476. Such instances may be accompanied by textual feedback which may display to the user that the electrodes have good contact 480-5 or prompt the user to either exfoliate the skin or press harder 480-6. The user interface may include a legend of overlays 490 for users to review which color or design features indicate predetermined configuration 472, achievement of correct configuration 473, actual electrode location 474, good electrode contact 475, and poor electrode contact 476.

2. Signal Recording/Data Cataloguing

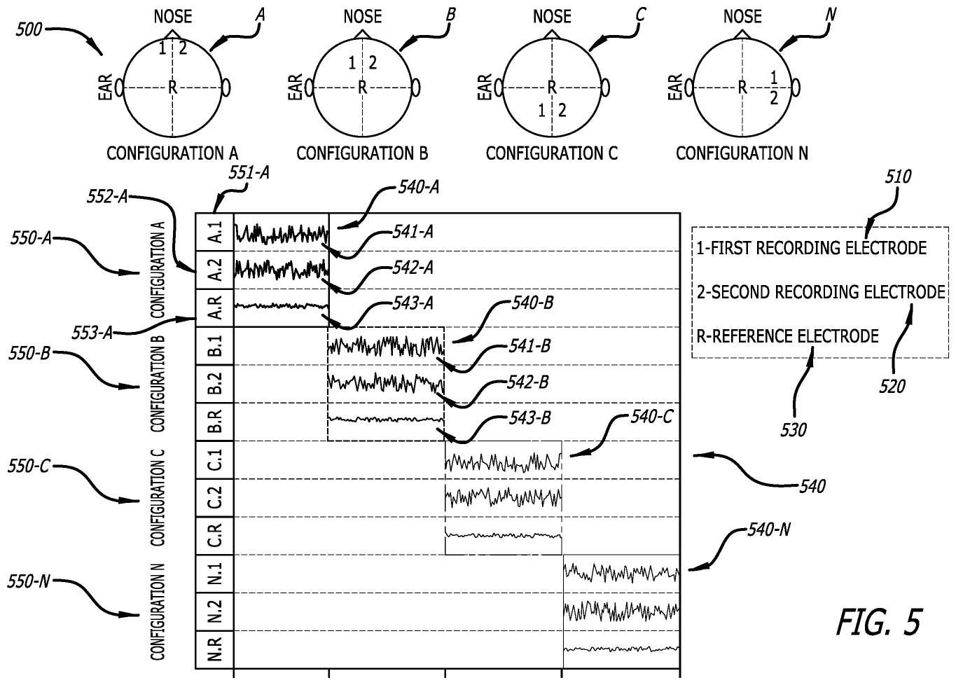

FIG. 5, refers to a methodology 500 to catalog acquired information from multiple unique configurations with accurate device positional coordinates and temporal information according to various embodiments described herein. In some embodiments, the cataloging and storage of data may be completely automated for efficiency. For example, the onset and conclusion of a sub-session may be characterized by variations in the user-applied force captured by force sensor 231 or by integrating the 3D coordinate from tracking units 230 and 240. In some implementations, the onset and culmination of the sub-session may be semi-automated where the user may either press a physical button on the device 100 with binary outputs or a prompt on the user-interface 400 to trigger the data cataloging and storage process. In some implementations, users may have the option to override the automated data cataloging process. An exemplary data cataloging and storage implementation 500 for an EEG session with the device that may be divided into N sub-sessions where N may be greater than or equal to one. For each sub-session, greater than one electrode 211 may be placed at unique configurations A, B, C and N to gain coverage of the subject's scalp. Data may be organized in a matrix 540 that is divided into sub-session matrices 540-A, 540-B, 540-C and 540-N that may act as independent units to store information from associated sub-sessions obtained at step 331. Each of the sub-session matrices 540-A, 540-B, 540-C and 540-N may be cataloged with a label 550-A, 550-B, 550-C and 550-N that indicates an overall coordinate value of the configuration. For example, if device 100 is configured with three mechanical extensions holding electrodes at distal end, the overall coordinate may be the centroid of the three electrodes when arranged in a configuration on the subject's body. The centroid of the three electrodes may coincide with the position of the barrel with respect to the mechanical extensions. Similarly, the overall coordinate value may be calculated as the center of the deployed configuration for other exemplary embodiments with N number of mechanical extensions. The sub-session matrices may be further divided into multiple 1D rows or columns to store temporal information. The sub-session matrix for configuration A 540-A may be divided into rows or columns 541-A, 542-A, 543-A and sub-session matrix for configuration B 540-B may be divided into rows or columns 541-B, 542-B, 543-C. For example, a recording session has N sub-sessions with multiple recording electrodes 510 and 520 and a reference electrode 530. At the first sub-session where the combination of electrodes 510, 520 and 530 is positioned at A, the information may be stored in 1D rows or columns at 541-A, 542-A and 543-A respectively. The 1D rows or columns 541-A, 542-A and 543-A may be labeled with coordinate values 551-A, 552-A and 553-A specific to the electrode 510, 520 and 530 that performed the action. Similar procedures can be repeated for the subsequent sub-sessions 540-B, 540-C and 540-N and cataloged with a coordinate information label 550-B, 550-C and 550-N. The organization of the information collected from the rapid positioning system through the process 300 into matrix, sub-matrices and 1D rows or columns may allow for efficient storage of metrics including spatial coordinates of configurations, temporal information of the sub-sessions and process related data. Such automated sorting and cataloging of data mitigate the operational workload for users, reduces errors in data storage and reinforces reproducibility amongst different users and subjects. The structured organization of the information may assist the medical practitioner during future data review and enable the process to provide consistent inputs to a machine learning algorithm for future advancements.

### IV. ANOMALY PREVENTION

The present disclosure also includes embodiments of systems, components and methods for automatically assessing and validating the electrode configuration, ensuring the optimal electrode contact with a dynamic feedback loop for maximum user engagement and understanding. The methodology disclosed enables acquisition of multiple biosignals (EEG, ECG and EMG) or provide electrical stimulation to heart, brain or a muscle group through a single rapid positioning system.

In some embodiments, the system prevents recording of erroneous biosignal values by monitoring signal strength and the force applied to the skin surface. In cases where both metrics may proportionally contribute to performance deterioration, both suggestions to exfoliate and adjust user-applied force may be prompted through any of the aforementioned feedback methodologies. The final skin contact results may be displayed to the user on the user interface of the output tablet before proceeding 324.

A. Proper Signal Quality

If poor electrode-impedance is measured, the user may be prompted to exfoliate the skin, remove the subject's hair from the area of interest or complete other necessary steps to achieve the correct contact 327. Feedback in such instances may be provided through LED 212, haptic feedback 212, visual cues 470, or textual cue 480. Scalar electrode-impedance values may be divided into excellent, good, or poor categories based on the type of electrode used. For example, in case of a wet electrode setup good contact may be categorized by impedance values under 10 kiloohms (kΩ) and excellent under 5 kΩ. In some instances, the signal quality may be poor due to low device battery or high internal device temperatures. Feedback in such instances may be provided through LED 212, haptic feedback 212, visual cues 470, or textual cue 480. For example, the text cue 480 could display a warning “Low device battery” or “High electronics temperature” to the operator, prompting the operator to pause signal acquisition and avoid low quality data acquisition.

B. Proper Signal Strength

A force sensor 231 may be enclosed in the electrode tracking unit 230 at appropriate locations on the device 100 and 110 to record the force experienced by the electrode when a user force is applied on or removed from the subject. The system may be designed such that the force sensor may be connected to the non-recording end or adjacent to the electrode 211. Recorded force values will be categorized in multiple levels such as inadequate, optimal or excessive based on defined thresholds.

If inadequate, excessive or highly varying force sensor values are observed between multiple electrode units 210 based on predetermined parameters that suggest insufficient or inconsistent user-applied force, a visual feedback may be provided to the user through an LED 212, haptic feedback 212, visual cues 470 or textual cue 480 on the output tablet for readjustments 326.

In some implementations, the quality of contact may be calculated based on the output values of the force sensors 231. Low force sensor values may indicate that the electrodes 211 have insufficient contact against the subject's skin and require adjustment. Large differences in the force experienced by different electrodes can also create inconsistencies in the amount of skin-contact and subsequently the quality of acquired signal. The quality of contact may also be tested by measuring electrode-skin impedance to ensure sensitive bioelectric signals do not deteriorate at the recording sites. For example, in some embodiments, the user-applied force may be monitored at a 2 Hz frequency to prevent prolonged electrode lift off and validate the electrode-contact information. This may prevent the overall degradation of electrode performance or dramatic mismatch in performance between different electrodes.

### Computer Systems

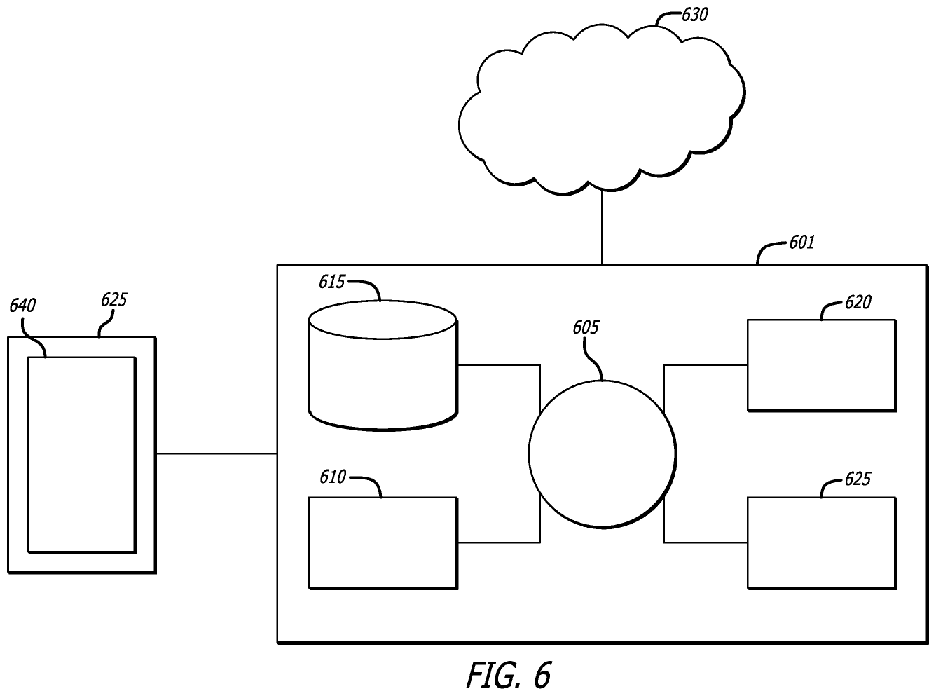

The present disclosure provides computer systems that are programmed to implement methods of the disclosure. FIG. 6 depicts a computer system 601 that is programmed or otherwise configured as a component of electrode positioning systems disclosed herein and/or to perform one or more steps of methods of positioning one or more electrodes and/or cataloguing electrical signals disclosed herein. The computer system 601 can regulate various aspects of automated of the present disclosure, such as, for example, assisting with proper positioning of electrodes, recording obtained electrical signals, and verifying proper electrode placement and sample quality disclosed herein. The computer system 601 can be an electronic device of a user or a computer system that is remotely located with respect to the electronic device. The electronic device can be a mobile electronic device. An output tablet, as referred to herein, may be any computing device compatible with the systems disclosed herein.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries, and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., a mediator computer). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, information related to the positioning of electrodes. Examples of UI's include, without limitation, a graphical user interface (GUI) and web-based user interface.

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

Although the above steps show the method of placing the rapid positioning system and validating its configuration with respect to the subject's body, variations of the presented disclosure may be implemented to achieve similar results. The steps in the methodology may be added, deleted, completed in different order or have sub-steps. Some of the steps to accurately validate the configuration of the system with respect to the subject's body may be repeated multiple times to achieve precision. Similarly, the methodology to finalize predetermined configuration for a suspected diagnosis and catalogue data with associated biosignal or electro-stimulation may be altered to achieve similar outcomes. Some rapid positioning systems can function without position validation or accurate cataloging in real-time and lacking details can be manually added post-hoc.

### VI. EXAMPLES

The following examples are included for illustrative purposes only and are not intended to limit the scope of the invention.

### Example 1: Monitoring for Seizures

In some embodiments, if a medical practitioner suspects that a subject may be suffering from seizures, the practitioner can use a two-recording channel rapid positioning system to screen multiple configurations on the brain in a short amount of time and derive conclusive results. Such techniques may follow a process of elimination where the simplest conditions are screened first by monitoring the suggested configurations for short epochs of time. The proposed set of electrode positions may be pre-selected in the most predominant brain regions for the ailment based on user-input at the modified step 311. Seizures can persist in both hemispheres of the brain (bilateral) or one hemisphere (unilateral). As bilateral activity persists in the hemispheres, the user may place the device with two recording electrodes at a bi-hemispheric configuration across the midsagittal plane (e.g.: F3 and F4) to detect any seizure activity. Quantification of seizure activity may be automated in some cases using various signal processing, statistical and entropy parameters. If bilateral activity is observed at the first configuration, the output interface may prompt additional configurations symmetric to the midsagittal plane on the subject's scalp to confirm the activity (e.g.: P3 and P4). If only unilateral activity is observed at a first configuration, the subsequent hemisphere-specific configurations may be displayed to the user for localizing the source of abnormal activity (e.g.: F3 and P3 for left hemisphere and F4 and P4 for right hemisphere). If no abnormal activity is observed at a first configuration, the outcome may be validated by monitoring additional hemisphere-specific or non-hemisphere-specific configurations at a user's discretion.

While the importance and methodology to screen seizures is discussed in detail as an example above, the proposed screening technique may be modified to match the needs of other ailments as well. The present disclosure is not limited to a specific ailment, the number of electrodes or the example of configuration. The additional configurations to be monitored can be greater than one and the process of ruling out a bilateral versus unilateral activity can be modified based on subject's presented clinical signs or medical history. In addition, the predetermined configurations can also be provided by an experienced medical professional through telemedicine, or any other communication method, to a fellow medical professional to undertake the screening procedure.

### Example 2: Concussion Screening and Management

Athletes and sports trainers who are engaged in contact-sports can use a two-recording channel rapid positioning system to objectively screen and manage concussions or mild-traumatic brain injury. Athletes may self-record pre-game baseline when they are head-injury free. In case there is a head injury during the game, a sports trainer may use the rapid positioning system for an on-site concussion assessment and the athletes can continue recovery monitoring through self-administration of the device before returning to play. The recorded EEG may be further analyzed using quantitative EEG (qEEG) techniques that heavily rely on the accuracy of electrode configuration positions and optimal signal quality. Rapid positioning systems may dramatically reduce the EEG device training period for athletes and increase technology accessibility while ensuring the EEG data is collected from correct electrode configurations with optimal signal quality. Rapid positioning systems ensure reliable and reproducible signals are capture regardless of the different levels of device experience amongst operators.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system to guide placement of one or more electrodes on a surface of a subject to detect bioelectrical signals, the system comprising:
one or more inertial sensors coupled with the one or more electrodes; and
a computing device comprising a) a processor in operative communication with the one or more inertial sensors, and b) a non-transitory computer readable storage medium having a computer program with instructions executable by the processor, such that the processor is configured to: i) calibrate a first position of the one or more electrodes to set an origin location, ii) receive data from the one or more inertial sensors to track a second position of the one or more electrodes relative to the origin location, and iii) output a feedback corresponding to the second position of the one or more electrodes.

2. The system of claim 1 further comprising a camera in operative communication with the processor, and wherein the processor is configured to receive data from the camera, via the computer program instructions, such that the processor is configured to either (i) track the second position of the one or more electrodes and output the feedback based on the one or more inertial sensors and the camera or (ii) track the second position of the one or more electrodes based on a position of the at least one edge in the one or more images.

3. The system of claim 2, further comprising one or more force sensors to detect a force being applied by the one or more electrodes to the surface, and wherein the processor, via the computer program instructions, is configured to output the feedback based on the force being applied by the one or more electrodes to the surface.

4. The system of claim 1, wherein the processor outputs the feedback based on a strength of the bioelectrical signals detected by the one or more electrodes.

5. The system of claim 1 further comprising an output interface configured to display an electrode overlay, wherein the origin location and the second position of the one or more electrodes are visualized onto the electrode overlay.

6. The system of claim 5, wherein the electrode overlay comprises a predetermined configuration overlay to depict one or more desired electrode placement locations, and wherein the feedback corresponds to the second position of the one or more electrodes relative to the one or more desired electrode placement locations.

7. The system of claim 5, wherein the electrode overlay is generated based on a head width of the subject, a head length of the subject, a head curvature of the subject, or a combination thereof.

8. The system of claim 1, wherein the processor, when executing the computer program instructions, is configured to catalogue (i) the bioelectrical signals detected by the one or more electrodes or (ii) the second position of the one or more electrodes corresponding to the bioelectrical signals received by the one or more electrodes.

9. The system of claim 1 further comprising a camera in operative communication with the processor, wherein the processor is configured to receive data from the camera, track the second position of the one or more electrodes, and output the feedback based on the one or more inertial sensors and the camera.

10. The system of claim 9, wherein the processor, upon execution of the computer program instructions, is configured to detect at least one edge of the surface from one or more images captured by the camera, and track the second position of the one or more electrodes based on a position of the at least one edge in the one or more images.

11. The system of claim 5, wherein the electrode overlay comprises a predetermined configuration overlay to depict one or more desired electrode placement locations, and wherein the feedback corresponds to the second position of the one or more electrodes relative to the one or more desired electrode placement locations.

12. The system of claim 11, wherein the electrode overlay is generated based on a head width of the subject, a head length of the subject, a head curvature of the subject, or a combination thereof of a subject

13. A computer implemented method of guiding placement of one or more electrodes on a skin surface of a subject, comprising:
a) calibrating an initial position of the one or more electrodes;
b) tracking a deviation of a second position of the one or more electrodes relative to the initial position using one or more sensors; and
c) providing a feedback based on the deviations.

14. The method of claim 13, wherein the one or more sensors comprise an inertial sensor, an image sensor, or a combination thereof.

15. The method of claim 13 further comprising visualizing an origin position and the second position on the surface through an electrode overlay.

16. The method of claim 13 further comprising:
setting an origin on the skin surface constituting the initial position; and
determining one or more desired electrode placement locations relative to the origin.

17. The method of claim 13, wherein the calibrating of the initial position comprises applying an edge detection algorithm via a computing device in operative communication with the one or more sensors.

18. The method of claim 13, wherein the providing of the feedback is based on a strength of one or more electrical signals detected by the one or more electrodes.

19. The method of claim 13 further comprising visualizing an origin position and the second position on the skin surface through an electrode overlay, the electrode overlay comprises a predetermined configuration overlay to depict one or more desired electrode placement locations, and wherein the feedback corresponds to the second position of the one or more electrodes relative to the one or more desired electrode placement locations.

20. The method of claim 19, wherein the electrode overlay is generated based on a head width of the subject, a head length of the subject, a head curvature of the subject, or a combination thereof.

21. The method of claim 13 further comprising:
cataloguing of the one or more electrical signals.

22. The method of claim 21, wherein the one or more sensors comprise an image sensor, and wherein the method further comprises cataloguing images obtained by the image sensor.

23. The method of claim 13, wherein the tracking of the deviation comprises detecting deviations from the initial position by at least receiving data from one or more sensors being one or more inertial sensors, the one or more inertial sensors comprise at least one (i) accelerometer or (ii) gyroscope.

24. The method of claim 13, wherein the tracking of the deviation comprises receiving data from one or more inertial sensors, the one or more inertial sensors comprise at least one (i) accelerometer or (ii) gyroscope.

25. The method of any one of claim 13 further comprising:
enabling a subject to self-administer an EEG analysis, ECG analysis, EMG analysis, or a combination thereof.

26. The method of claim 19, wherein the tracking of the deviation comprises receiving data from a camera.

27. The method of claim 26, wherein the detecting of the deviation further comprises detecting the origin with the camera and evaluating the position of the origin in a series of images captured by the camera.

28. A system to guide placement of one or more electrodes on a surface of a subject to detect bioelectrical signals, the system comprising:
one or more sensors coupled with the one or more electrodes;
a computing device comprising a) a processor in operative communication with the one or more inertial sensors, and b) a non-transitory computer readable storage medium having a computer program with instructions executable by the processor, such that the processor is configured to: i) calibrate a first position of the one or more electrodes to set an origin location, ii) receive data from the one or more sensors to track a second position of the one or more electrodes relative to the origin location, and iii) output a feedback corresponding to the second position of the one or more electrodes; and
an output interface configured to display an electrode overlay, wherein the origin location and the second position of the one or more electrodes are visualized onto the electrode overlay and the electrode overlay is generated based on a head width of the subject, a head length of the subject, a head curvature of the subject, or a combination thereof.
